(19) United States
(12) Patent Application Publication
(10) Pub. No.: US 2017/0119791 A1
(43) Pub. Date: May 4, 2017

(54) METHODS AND COMPOSITIONS FOR DERMATOLOGICAL USE COMPRSING BETAMETHASONE AND BIOPOLYMERS

(71) Applicant: APEX LABORATORIES PRIVATE LIMITED, Chennai (IN)

(72) Inventors: Sular Subramaniam Vanangamudi, Chennai (IN); Srinivasan Murali, Chennai (IN); Srinivasan Madhavan, Chennai (IN)

(21) Appl. No.: 15/368,554

(22) Filed: Dec. 2, 2016

Related U.S. Application Data
(63) Continuation-in-part of application No. PCT/IB2016/053255, filed on Jun. 3, 2016.
Application No. PCT/IB2016/053255 is a continuation-in-part of application No. PCT/IB2016/053256, filed on Jun. 3, 2016.

(30) Foreign Application Priority Data
Jun. 10, 2015 (IN) 2889/CHE/2015
Jun. 10, 2015 (IN) 2890/CHE/2015

(51) Int. Cl. A61K 31/573; A61K 9/06; A61K 47/36; A61K 47/08; A61K 47/12; A61K 47/10; A61K 47/14; A61K 9/00; A61K 47/44

(52) U.S. Cl. CPC A61K 31/573; A61K 9/0014; A61K 9/06; A61K 47/36; A61K 47/44; A61K 47/12; A61K 47/10; A61K 47/14; A61K 47/08

(57) ABSTRACT

Disclosed are compositions comprising betamethasone, such as betamethasone dipropionate or betamethasone valerate, and a biopolymer in a cream base, wherein the cream base comprises a primary and a secondary emulsifier, a waxy material, a co-solvent, a preservative, an acid, a chelating agent, a buffering agent, and water. The biopolymer comprises chitosan. The compositions disclosed herein are suitable for the treatment of dermatological conditions including but not limited to healing wounds and treatment of dermatitis.

### CROSS-REFERENCE TO RELATED APPLICATION

This application is a continuation-in-part of PCT/IB2016/053255, filed Jun. 3, 2016, which application in turn claims priority from Indian Provisional Application Serial 2889/CHE/2015, filed Jun. 10, 2015, the contents of which are incorporated herein by reference. This application is also a continuation-in-part of PCT/IB2016/053256, filed Jun. 3, 2016, which application in turn claims priority from Indian Provisional Application Serial 2890/CHE/2015, filed Jun. 10, 2015, the contents of which are incorporated herein by reference.

### FIELD OF THE INVENTION

This present invention is related to dermatological compositions comprising topical corticosteroids and a biopolymer, wherein said biopolymer comprises chitosan.

### BACKGROUND OF THE INVENTION

The outer layer of skin surrounding the body performs an important protective function as a barrier against infection, and serves as a means of regulating the exchange of heat, fluid and gas between the body and external environment. When skin is removed or damaged by being abraded, burned or lacerated, this protective function is diminished. Areas of damaged skin are conventionally treated with dermatological agents and protected by the application of wound dressings to facilitate wound healing.

Wounds to skin and the underlying tissues of animals may be caused by a multitude of external insults such as friction, abrasion, laceration, burning or chemical irritation. Damage to such tissues may also result from internal metabolic or physical dysfunction, including but not limited to bone protrudence, diabetes, circulatory insufficiencies, or inflammatory processes. Normally tissue damage initiates physiological processes of regeneration and repair. In broad terms, this process is referred to as the wound healing process.

Wound healing, or wound repair, is the body's natural process of regenerating dermal and epidermal tissue. The wound healing process is normally uneventful and may occur regardless of any intervention, even in the case of acute or traumatic wounds. However, in certain situations where an underlying metabolic condition or perpetual insult such as pressure is a contributing factor, the natural wound healing process may be retarded or completely arrested, resulting in a chronic wound. When an individual is wounded, a set of complex biochemical events takes place in a closely orchestrated cascade to repair the damage.

The wound healing process progresses through distinct stages leading to the eventual closure, and restoration of the natural function of the tissues. Injury to the skin initiates an immediate vascular response characterized by a transient period of vasoconstriction, followed by a more prolonged period of vasodilation. Blood components infiltrate the wound site, endothelial cells are released, exposing fibrillar collagen, and platelets attach to exposed sites. As platelets become activated, components are released which initiate events of the intrinsic coagulation pathway. At the same time, a complex series of events trigger the inflammatory pathways generating soluble mediators to direct subsequent stages of the healing process.

Wound healing is a complicated process that recruits at least four distinct cell types. Though the process is continuous, it is commonly referred to as occurring in “phases.” The main phases of wound healing include coagulation, which begins immediately after injury; inflammation, which initiates shortly thereafter; a migratory and proliferate process, which begins within days and includes the major processes of healing and a remodeling process, which may last for up to a year and is responsible for scar tissue formation and development of new skin.

Coagulation performs its function of hemostasis, initiating healing and leaving behind messengers that bring on an inflammatory process. Inflammation protects the wound from infection and leaves behind its own set of messengers, important signals that bring on the migration and proliferation of macrophages, lymphocytes, fibroblasts, keratinocytes and endothelial cells. In the next phase fibroblasts become dominant and a collagenous matrix is deposited. Finally, there is a remodeling process that aims to restore full and normal structure. Each of these components plays a specific and irreplaceable role in the continuum of healing. A delay in, or absence of any one can result in a prolongation or even a prohibition of healing.

Wound healing is a multifaced physiological process affected by several factors. These include local factors (growth factors, edema and ischemia, low oxygen tension, and infection), regional factors (arterial insufficiency, venous insufficiency and neuropathy), systemic factors (inadequate perfusion and metabolic disease) and other miscellaneous factors, such as nutritional state, preexisting illnesses, exposure to radiation therapy and smoking. In general, chronic wounds may be managed by preventing or medically treating infections through debridement and occlusive dressings. For wounds that are unresponsive to such interventions, the use of skin replacements may be a viable option.

Given the complex interplay of multiple phases and components in wound healing, it is not surprising that many factors affecting the healing process have been identified. Recognizing and understanding such factors may lead to improved clinical management of recalcitrant or chronic wounds. Patients with risk factors for wound healing may be identified and treated more aggressively or may be better managed for prevention of infection and/or non-healing wounds. Factors affecting wound healing fall into several categories, based on their source; local, regional or systemic.

Trends in modern medical practices have shown that the wound healing of both acute and chronic wounds may be significantly improved by clinical intervention using methods and materials that optimize wound conditions to support the physiological processes of the progressive stages of wound healing. Key factors in providing the optimal conditions are the prevention of scab formation, the prevention of infection and the maintenance of an optimal level of moisture in the wound bed.

In addition to treatment of wounds, several dermatological conditions exist that require therapeutic attention. Such conditions include, for example, acne and related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis (eczema), contact dermatitis, dermatitis herpetiformis, generalized exfoliative dermatitis, seborrheic dermatitis, rosacea, shingles, sweating disorders, vitiligo and viral skin disease. Of particular interest is dermatitis, generally considered an inflammation of the skin that is characterized by skin that may be red, swollen, blistered, scabbed, scaly, oozing, or itchy. Whereas some types of dermatitis are caused by allergies, a majority of dermatitis cases do not have any known causes.

The term dermatosis generally refers to diseases of the integumentary system. This classification includes everything on the surface of the body: skin, nails, and hair. Any condition affecting the skin could be categorized a dermatosis. This doesn't include skin conditions that involve inflammation (that would be dermatitis). Skin is the largest organ on the human body and thousands of documented conditions can affect the skin, hair, and nails. As discussed, skin has several layers, including the epidermis, the dermis, and the subcutaneous tissue. A dermatosis may involve changes in any or all of these skin layers. Terms associated with dermatosis of the skin include: rash (a wide variety of skin conditions that are red and raised), lesion (an area of skin that is abnormal), macule (a change in color or consistency of the skin), papule (a bump on the skin smaller than 1 cm in diameter), nodule (a bump on the skin larger than 1 cm in diameter), plaque (a large area of affected skin with defined edges that may flake or peel), vesicles and bullae (raised bumps that are filled with fluid), lichenification (a thick discoloration of skin, such as lichen on a tree), and pustules (a bump that contains pus, possibly due to infection).

Some of the most common forms of dermatosis include: acne (when the oil glands in skin cause pimples and scarring), impetigo (a skin infection caused by bacteria), melanoma (the most serious form of skin cancer), basal cell carcinoma (the most common form of skin cancer that strikes in the top layer of the epidermis), moles (dark growths on the skin) actinic keratosis (crusty pre-cancerous growths caused by sun damage), erythema nodosum (inflammation of fat under the skin of the shins, resulting in red lumps), lupus erythrematosus (an autoimmune disease that may create a “butterfly” rash on the face), morphea (localized scleroderma, or hardened patches of skin), vitiligo (white of patches of skin), tinea (fungal infection of skin that leaves round marks), nail clubbing (when nails curve around the fingertips due to low oxygen levels in the blood), spoon nails (koilonychias—an indication of iron deficiency or liver condition called hemochromatosis), onycholysis (when the fingernails become loose and separate from the nail bed), Beau's lines (indentations that run across the nails), yellow nail syndrome: a discoloration of the nails, alopecia areata (hair loss in round patches) and wrinkles (the influence of aging on skin). The most common causes of dermatosis include: autoimmune disorders, bacterial/fungal/viral infection or genetic susceptibility.

Dermatological pharmacology is the study of agents and their actions in an abnormal dermatological or wound environment. Dermatological pharmacology generally comprises three classes of agents: drugs, biologics and special biologics such as those produced by biotechnology. Currently available treatments for both topical and systemic treatment of dermatological issues typically employ corticosteroids in a base component.

There continues to be a need for improved therapeutics that not only address wound healing and repair, but also treat pain associated with dermatological problems, and therapeutics that reduce inflammation, infection, scarring and overall discomfort. There is also a need for therapeutics for dermal conditions that are easily used and applied by patients to accommodate treatment times that may be long and extended. Ideal treatment modalities for wounds and dermal pathologies should be effective and sufficiently straightforward so that a high degree of patient compliance is achieved. Furthermore, there is a need for improved therapeutics requiring a simple and relatively short duration of administration. There is also a need for effective topical treatment of dermatological conditions wherein the compositions enable successful penetration of the active agent, preferably, effective treatments include the penetration, accumulation and maintenance of an effective concentration of active agent at the site of the wound or skin lesion. Compositions are also needed that are effective for treatment pathological conditions in the skin and dermal structures.

### SUMMARY OF THE INVENTION

Disclosed herein are novel methods and compositions comprising betamethasone, including betamethasone dipropionate and betamethasone valerate, and a biopolymer in a cream base, wherein the cream base comprises a primary and a secondary emulsifier, a waxy material, a co-solvent, a preservative, an acid, a chelating agent, a buffering agent, and water. In certain aspects, the biopolymer comprises a chitosan component. In certain aspects, the chitosan component comprises an unbranched binary polysaccharide consisting of two units N-acetyl-D-glucosamine and D-glucosamine used for the treatment of skin regeneration and rejuvenation and wound healing.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 provides graphical depiction of the formation of a film when using the formulation of the present invention. FIG. 1A corresponds to betamethasone dipropionate and FIG. 1B corresponds to betamethasone valerate.

### DETAILED DESCRIPTION

Before the present compounds, compositions, articles, devices, and/or methods are disclosed and described, it is to be understood that they are not limited to specific synthetic methods or specific pharmacology methods unless otherwise specified, or to particular reagents unless otherwise specified, as such may, of course, vary. It is also to be understood that the terminology used herein is for the purpose of describing particular embodiments only and is not intended to be limiting.

### A. Definitions

1. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

3. In this specification and in the claims which follow, reference will be made to a number of terms which shall be defined to have the following meanings:

4. “Optional” or “optionally” means that the subsequently described event or circumstance may or may not occur, and that the description includes instances where said event or circumstance occurs and instances where it does not.

5. Throughout this application, various publications may be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

6. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular formulation is disclosed and discussed and a number of modifications that can be made to a number of active agents including the biopolymer are discussed, specifically contemplated is each and every combination and permutation of the formulation and the modifications that are possible unless specifically indicated to the contrary. Thus, if a class of active agents A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

7. The present invention comprises the use of biopolymers, including, but not limited to chitin, chitosan, chitosan derivatives chitosan related materials both naturally occurring and synthetically produced.

8. Chitosan is a biopolymer with skin regeneration and rejuvenation properties due to its unique physical nature. Chitosan acts as a biocatalyst in accelerating wound healing. Due to its positive charge it couples with negatively charged blood cells and aids in clotting of blood. Chitosan also contributes to controlling microbial mobility because of its charge and prevents spread of infections. As a micro-film forming biomaterial, chitosan helps in reducing the width of a wound, controls the oxygen permeability at the wound site, and absorbs wound discharge, which is very much essential for faster wound healing. It also reduces itching by providing a soothing effect.

9. Chitosan is an un-branched binary polysaccharide consisting of two units N-Acetyl-D-glucosamine and D-glucosamine linked in β (1,4) manner. The chemical name of chitosan is Poly-β-(1,4)-2-Amino-2-deoxy-D-glucose. In certain aspects, chitosan is used as a film forming, mucoadhesive and viscosity-increasing agent. In certain other aspects, chitosan is also used as a binder and disintegrating agent in tablet formulations. Chitosan generally absorbs moisture from the atmosphere or environment and the amount absorbed typically depends upon the initial moisture content, temperature and relative humidity of the environment. Chitosan is regarded as a non-toxic and non-irritant material. It is biocompatible with both healthy and infected skin and has been shown to be biodegradable.

10. In certain aspects, chitosan is produced commercially by deacetylation of chitin, which is the structural element in the exoskeleton of crustaceans (including but not limited to crabs, shrimp, lobsters, krill, woodlice, and barnacles, i.e. members of the Pancrustacia claude) and cell walls of fungi. The degree of deacetylation (% DD) can be determined by NMR spectroscopy, and the % DD in commercial chitosans ranges from 60 to 100%. On average, the molecular weight of chitosan as used herein is between 300,000 to 2,000,000 Daltons. A common method for the synthesis of chitosan is the deacetylation of chitin using sodium hydroxide in excess as a reagent and water as a solvent. The reaction occurs in two stages under first-order kinetic control. Activation energy for the first step is higher than the second; its value is an estimated 48.76 kJ/mol at 25-120 degrees C. This reaction pathway, when allowed to go to completion (complete deacetylation) yields up to 98% product.

11. The amino group in chitosan has a pKa value of approximately 6.5, which leads to a protonation in acidic to neutral solution with a charge density dependent on pH and the % DA-value. This makes chitosan water-soluble and a bioadhesive which readily binds to negatively charged surfaces such as mucosal membranes. In certain novel embodiments of the present invention, chitosan enhances the transport of pharmaceutical agents across epithelial surfaces, and is biocompatible and biodegradable. Purified quantities of chitosans are suitable for biomedical applications.

12. In certain novel embodiments of the present invention, chitosan and its derivatives, such as trimethylchitosan (where the amino group has been trimethylated), may be used in nonviral gene delivery. Trimethylchitosan, or quaternised chitosan, has been shown to transfect breast cancer cells, with increased degree of trimethylation increasing the cytotoxicity; at approximately 50% trimethylation, the derivative is the most efficient at gene delivery. Oligomeric chitosan derivatives (3-6 kDa) are relatively nontoxic and have good gene delivery properties.

13. Chitosan's properties allow it to rapidly clot blood, and has been granted approval in the United States and Europe for use in bandages and other hemostatic agents. Chitosan hemostatic products also reduce blood loss in comparison to gauze dressings and increase patient survival. Chitosan is hypoallergenic and has natural antibacterial properties.

14. Though not wishing to be bound by the following theory, it is thought that chitosan's hemostatic properties also allow it to reduce pain by blocking nerve endings. Chitosan hemostatic agents are often chitosan salts made from mixing chitosan with an organic acid (such as succinic or lactic acid). The hemostatic agent works by an interaction between the cell membrane of erythrocytes (negative charge) and the protonated chitosan (positive charge) leading to involvement of platelets and rapid thrombus formation. In certain embodiments, chitosan salts can be mixed with other materials to make them more absorbent (such as mixing with alginate), or to vary the rate of solubility and bioabsorbability of the chitosan salt. The chitosan salts are biocompatible and biodegradable making them useful as absorbable haemostats. Protonated chitosan is broken down by lysozyme in the body to glucosamine and the conjugate base of the acid (such as lactate or succinate) are substances naturally found in the body.

15. In certain embodiments, the disclosed compositions and methods of the present invention utilize chitosan's properties to allow it to be used in transdermal drug delivery; it is mucoadhesive in nature, reactive (so it can be produced in many different forms), and importantly, has a positive charge under acidic conditions. This positive charge comes from protonation of its free amino groups. Lack of a positive charge means chitosan is insoluble in neutral and basic environments. However, in acidic environments, protonation of the amino groups leads to an increase in solubility. The implications of this are very important to biomedical applications. This molecule uniquely maintains its structure in a neutral environment, but will solubilize and degrade in an acidic environment.

16. As described herein, chitin and chitosan (CS) are biopolymers having immense structural possibilities for chemical and mechanical modifications to generate novel properties, functions and applications especially in biomedical area. However, despite the availability and utility of chitosan, the actual utilization of chitin has been restricted by its intractability and insolubility until now. The present inventors have discovered and reduced to practice for the first time, novel compositions and methods of using chitin and chitosan for biomedical use, including but not limited to methods of treating a myriad of dermatological conditions.

17. The novel compositions of the present invention comprise the use of corticosteroids, including but not limited to topical corticosteroids well known to those skilled in the art. Such corticosteroids include for example, hydrocortisone, hydrocortisone acetate, cortisone acetate, diflorasone diacetate, tixocortol pivalate, prednisolone, methylprednisolone, prednisone, triamcinolone acetonide, triamcinolone alcohol, mometasone amcinonide, budesonide, desonid, fluocinonide, fluocinolone acetonide, halcinonide, betamethasone, betamethasone dipropionate, betamethasone valerate, betamethasone sodium phosphate, dexamethasone, dexamethasone sodium phosphate, fluocortolone, mometasone furoate, corticosteroid esters, halogenated corticosteroids (hydrocortisone-17-valerate, halometasone, halobestol propionate, alclometasone dipropionate, prednicarbate, clobetasone butyrate, clobetasone-17-butyrate, clobetasol propionate clobetasol-17-propionate, fluocortolone caproate, fluocortolone pivalate, fluprednidene acetate) and labile prodrug esters (hydrocortisone-17-butyrate, hydrocortisone-17-aceponate, hydrocortisone-17-buteprate, ciclesonide and prednicarbate). In certain aspects, the present invention comprises the use of inhalable steroids, including but not limited to flunisolide, fluticasone furoate, fluticasone propionate, triamcinolone acetonide, beclomethasone dipropionate, and budesonide. As is known those skilled in the art, certain corticosteroids may be suitable for topical, inhalation, oral, or systemic use including for example, intravenous and parenteral routes.

18. Though not wishing to be bound by the following theory, it is thought that corticosteroids act by the induction of phospholipase A₂ inhibitory proteins, collectively called lipocortins. It is postulated that these proteins control the biosynthesis of potent mediators of inflammation such as prostaglandins and leukotrienes by inhibiting the release of their common precursor Arachidonic acid. Arachidonic acid is released from membrane phospholipids by phospholipase A2.

19. Topical corticosteroids are classified by potency, ranging from weak to extremely potent. They include weak potent steroids, moderate potent steroids, potent steroids, very potent steroids and extremely potent steroids. The high potency steroids include betamethasone dipropionate, betamethasone valerate, diflorasone diacetate, clobetasol propionate, halobetasol propionate, desoximetasone, diflorasone diacetate, fluocinonide, mometasone furoate, triamcinolone acetonide, etc. low potency topical steroids include desonide, fluocinolone acetate, and hydrocortisone, etc. Topical corticosteroids are used for the relief of the inflammatory and pruritic manifestations of corticosteroid responsive dermatoses.

20. Betamethasone is a moderately potent glucocorticoid steroid with anti-inflammatory and immunosuppressive properties. Unlike other drugs with these effects, betamethasone does not cause water retention. It is applied as a topical cream, ointment, foam, lotion or gel to treat itching (e.g. from eczema). Betamethasone sodium phosphate is sometimes prescribed as an intramuscular injection for itching from various ailments including allergic reactions to poison ivy and similar plants. The compound is available as a number of ester derivatives: dipropionate (branded as Diprosone, Diprolene and others), valerate (branded as Betnovate, Celestone and others) and sodium phosphate.

21. Betamethasone dipropionate is a moderately potent steroid and is used as anti-inflammatory and antipruritic agent. It has the chemical name 9-fluoro-11β, 17,21-trihydroxy-16β-methylpregna-1,4-diene-3,20-dione 17,21-dipropionate, with the empirical formula C₂₈H₃₇FO₇, a molecular weight of 504.6 g/mol. Betamethasone dipropionate is a white to cream powder, insoluble in water and sparingly soluble in alcohol and freely soluble in acetone. Betamethasone dipropionate is a topical corticosteroid indicated for the relief of the inflammatory and pruritic manifestations of corticosteroid responsive dermatoses.

22. Betamethasone dipropionate is a corticosteroid with anti-inflammatory, antipruritic, and vasoconstrictive properties. Betamethasone dipropionate is thought to depress formation, release, and activity of endogenous mediators of inflammation, including prostaglandins, kinins, histamine, liposomal enzymes, and complement system, modifies body's immune response. In addition, betamethasone dipropionate is though to produce multiple glucocorticoid and mineralocorticoid effects.

23. Betamethasone dipropionate has been shown to have a wide range of inhibitory effects on multiple cell types (e.g. mast cells, eosinophils, neutrophils, macrophages and lymphocytes) and mediators (e.g. histamine, eicosanoids, leukotrienes, and cytokines) involved in inflammation and in the asthmatic response. These anti-inflammatory actions of corticosteroids may contribute to their efficacy in asthma and in skin lesions.

24. Betamethasone valerate is chemically designated as 9-Fluoro-11β, 17,21-trihydroxy-16β-methylpregna-1,4-diene-3,20-dione 17-valerate. The molecular formula and weight of Betamethasone valerate are C₂₇H₃₇FO₆ and 476.58 g/mol respectively; and 1.2 mg of betamethasone valerate is equivalent to 1.0 mg betamethasone. Betamethasone valerate is a white to practically white, odorless powder. It melts at 190° C. with decomposition. It is practically insoluble in water, freely soluble in acetone and in chloroform, soluble in alcohol, and slightly soluble in benzene and in ether. The exact mechanism of anti-inflammatory action of betamethasone valerate is unknown, however it produces multiple glucocorticoid and mineralocorticoid effects.

25. The extent of percutaneous absorption of topical corticosteroids is determined by many factors including, but not limited to, the vehicle, the integrity of the epidermal barrier, and the use of occlusive dressings. Topical corticosteroids may be absorbed from normal intact skin, and in addition, inflammation and/or other disease processes in the skin increase percutaneous absorption. Occlusive dressings substantially increase the percutaneous absorption of topical corticosteroids. Accordingly, an aspect of the present invention comprises the use occlusive dressings in combination with the novel compositions described herein as a valuable therapeutic adjunct for treatment of resistant dermatological conditions. Once absorbed through the skin, topical corticosteroids are handled through pharmacokinetic pathways similar to systemically administered corticosteroids. Corticosteroids are bound to plasma proteins in varying degrees, metabolized primarily in the liver and then excreted by the kidneys. Some topical corticosteroids and metabolites are also excreted into bile.

26. The pH value of human skin is somewhere between 4.5 and 6. Newborn baby's skin pH is closer to neutral (pH 7), but it quickly turns acidic. Nature has designed this probably to protect young children's skin, since acidity kills bacteria. As people age, skin becomes more and more neutral, and fewer bacteria is killed, hence the skin becomes weak and problematic. The pH value goes beyond 6 when a person actually has a skin problem or skin disease. In accordance with the foregoing, there is a preference for dermatological compositions to mirror a pH value closer to that of skin of a young adult.

27. The pH of the novel compositions described herein, comprising chitosan with betamethasone dipropionate or betamethasone valerate cream, is in the range from about 3 to 6. In contrast to available ointments, the presently claimed compositions are not greasy and are cosmetically elegant. In addition, because the active compound is preferably in an ionized form, transdermal penetration is more efficient and more effective.

28. The compositions disclosed herein are highly preferred because the design of the formulation enables active drug penetration of the skin resulting in optimum bio-dermal efficacy. The particle size of the active drug plays an important role here: not only must the particle size be such that therapeutic value is maintained, it must also be such that transdermal delivery is optimized. In a preferred aspect, the active drug is available in colloidal or molecular dispersed state. Also this is to be achieved in the safe pH compatible environment of skin (4.0 to 6.0). The novel compositions disclosed herein satisfy the stated parameters by incorporating optimal vehicles or co-solvents for the dissolution or dispersion of the drug. The disclosed compositions of the present invention are highly efficacious due to the pronounced anti-inflammatory and wound healing activity of the novel combination of the active ingredients, which are available in ultramicron-size, colloidal form, which enhance and enable effective skin penetration for therapeutic efficacy.

29. The novel compositions of the present invention are highly effective in protecting skin, regenerating skin, rejuvenating skin, as well controlling superficial wounds. Furthermore, the compositions of the present invention are particularly desirable as they are affordable, non-allergenic, and safe. In an embodiment, the novel compositions of the present invention comprise a unique combination of a topical corticosteroid (such as betamethasone), along with a biopolymer (such as chitosan).

30. In an embodiment, a betamethasone composition as described herein, such as betamethasone dipropionate or betamethasone valerate, provides rapid relief of pruritus (severe itching). In addition, novel betamethasone compositions of the present invention are also recommended for severe eczematic eruptions to provide instant relief to patients from itching and burning. Also monotherapy with betamethasone compositions assists in avoiding allergenic response to antifungals and antibacterials.

31. The present invention discloses novel and unique compositions comprising combinations of a steroid, betamethasone dipropionate, or betamethasone valerate with a biopolymer, chitosan. This novel combination is highly therapeutically effective as a result of the unique and desirable physical, chemical and therapeutic properties of chitosan with betamethasone dipropionate or betamethasone valerate. Though not wishing to be bound by the following theory, it is thought that the superior therapeutic efficacy of the compositions claimed herein result because chitosan functions as a film forming, biocompatible, non-allergenic biopolymer, protecting the skin by acting as a barrier, and betamethasone dipropionate/betamethasone valerate attenuates inflammation. Until the innovative discoveries by the present inventors, the unique combination of properties such skin protection, inhibiting the mobility of pathogens from one site to another, and other therapeutic advantages had not been realized. The present invention addresses this long felt need by incorporating the use of biopolymers such as chitosan to optimize skin protection (by way of film forming properties), immobilization of pathogenic microbes (due to its cationic electrostatic property) and wound healing.

32. As previously discussed herein, chitosan is a non-toxic and non-irritant material; it is biocompatible with both healthy and infected skin and has been shown to be biodegradable. In addition, chitosan shares certain chemical characteristics with GlycosAminoGlycans (GAGs), and GAGs like heparin, heparin sulfate, hyaluronic acid and keratin sulfate all are derivatives of 2-amino-2-deoxy-D-glucose which are present in many parts of human body. GAGs are essential building blocks of macromolecular frame work of connective and other tissues. It is believed that fetal wounds are known to heal without scars as a result of fetal skins being rich in hyaluronic acid. Chitosan/Polyglucosamine is structurally similar to hyaluronan and assists in wound healing with minimal scarring. Heparin enhances mitogen by induction and stabilization of fibroblast growth stimulating factor (FGF). Polyglucosamine may promote tissue growth and wound healing by forming complexes with heparin and acting to prolong the half-life of the growth factors.

33. As a film forming biomaterial, chitosan helps in reducing wound diameters and widths, controls oxygen permeability at the site, absorbs wound discharge and gets degraded by tissue enzymes thereby enabling healing at a faster rate. Chitosan also reduces itching by providing a soothing effect, and acts as a moisturizer.

34. The novel compositions disclosed herein are most stable and efficacious at ambient conditions and do not need special temperature control during transportation or storage, thereby making the present invention further desirable and versatile for a variety of uses including decreased maintenance considerations.

35. The present invention comprises novel compositions that not only diminish the possibility of infection, but also addresses the problem of arresting bleeding. Currently available products and therapies are less effective at controlling superficial bleeding and result in secondary and tertiary complications. The present invention simultaneously addresses bleeding, infection control and wound healing.

36. Disclosed herein are compositions comprising betamethasone compounds, including but not limited to betamethasone diproprionate, or betamethasone valerate, and a biopolymer in a cream base, wherein the cream base comprises a primary and a secondary emulsifier, a waxy material, a co-solvent, a preservative, an acid, and water. In an embodiment, the compositions further comprise an anti-oxidant, a chelating agent, a buffering agent, or a humectant. The betamethasone compound, betamethasone diproprionate or betamethasone valerate, may be added in an amount between 0.001% (w/w) and 10% (w/w), between about 0.01% (w/w) and 5% (w/w), or 0.001% (w/w) and 2% (w/w). The biopolymer may comprise chitosan and the chitosan may be added in an amount between 0.01% (w/w) and 5% (w/w) by weight, in an amount from 0.01% (w/w) to 1.5% (w/w), or 0.5%(w/w). In addition, the chitosan used in compositions disclosed herein may comprise a molecular weight in the range of 50 kDa to 5000 kDa. The primary and secondary emulsifiers of the disclosed compositions are selected from a group comprising cetostearyl alcohol, cetomacrogol-1000, cetyl alcohol, stearyl alcohol, isopropyl myristate, polysorbate-80, Span-80; and the primary and secondary emulsifiers may be present in the amount of 1% (w/w) to 25% (w/w). The waxy material of the disclosed compositions may be selected from a group comprising white soft paraffin, liquid paraffin, and hard paraffin; and wherein the waxy material is added in an amount from 5% (w/w) to 30% (w/w); and the co-solvent may be selected from a group comprising propylene glycol, hexylene glycol, polyethylene glycol-400; wherein the co-solvent is added in an amount from about 5% (w/w) to 50% (w/w). The acid of the disclosed compositions may be selected from a group comprising HCl, H₂SO₄, HNO₃, and lactic acid; and the acid may be added in an amount from about 0.005% (w/w) to 1% (w/w). The preservative of the disclosed compositions may be selected from a group comprising methylparaben, propylparaben, chlorocresol, potassium sorbate, benzoic acid, phenoxyethanol, and benzyl alcohol; and may be added in an amount from 0.02% (w/w) to 0.5% (w/w). The buffering agent of the disclosed compositions may be selected from the group comprising disodium hydrogen ortho phosphate, sodium hydrogen ortho phosphate; and may be added in an amount of 0.05% (w/w) to 1% (w/w). The disclosed compositions may further comprise water, wherein the water is purified water, and wherein the water is added in the range of 20% (w/w) to 75% (w/w), or 35% (w/w) to 60% (w/w). The anti-oxidants incorporated into the disclosed compositions may be selected from the group comprising butylated hydroxy anisole, butylated hydroxy toluene; wherein the anti-oxidant is added in an amount of 0.001% (w/w) to 5% (w/w); the chelating agents may be selected from the group comprising disodium EDTA; and may be added in an amount 0.05% (w/w) to 1% (w/w). The compositions may further comprise a humectant, wherein the humectant is selected from a group comprising glycerin, propylene glycol, sorbitol; and wherein the humectant is added in an amount of 5% (w/w) to 20% (w/w). Disclosed herein are methods for making compositions comprising the mixing of betamethasone, including but not limited to betamethasone proprionate or betamethasone valerate, and a biopolymer in a cream base, wherein the cream base comprises a primary and a secondary emulsifier, a waxy material, a co-solvent, a preservative, an acid, and water. The disclosed methods may further comprise a chelating agent, a buffering agent, anti-oxidant, or a humectant and a biopolymer comprising chitosan. The methods may involve the addition of betamethasone (betamethasone diproprionate, or betamethasone valerate) in an amount between 0.001% (w/w) and 10% (w/w), between about 0.01% (w/w) and 5% (w/w), or 0.001% (w/w) and 2% (w/w). The methods may involve the addition of chitosan in an amount between 0.01% (w/w) and 5% (w/w) by weight, in an amount from 0.01% (w/w) to 1.5% (w/w), or 0.5%(w/w).

Pharmaceutical Carriers/Delivery of Pharmaceutical Products

37. The disclosed compositions may be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

38. The disclosed compositions may be administered topically, transdermally, extracorporeally, or the like, including topical intranasal administration or administration by inhalant. The exact amount of the compositions required will vary from subject to subject, depending on the species, age, weight and general condition of the subject, the severity of the disorder being treated, its mode of administration and the like. Thus, it is not possible to specify an exact amount for every composition. However, an appropriate amount can be determined by one of ordinary skill in the art using only routine experimentation given the teachings herein.

39. The compositions can be used therapeutically in combination with a pharmaceutically acceptable carrier.

40. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

41. Pharmaceutical carriers are known to those skilled in the art. These most typically would be standard carriers for administration of therapeutic agents to humans, including solutions such as sterile water, saline, and buffered solutions at physiological pH. The compositions can be administered topically. Other compounds will be administered according to standard procedures used by those skilled in the art.

42. The disclosed compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

43. The disclosed compositions may be administered in a number of ways depending on whether local or systemic treatment is desired, and on the area to be treated. Administration may be topically (including ophthalmically, vaginally, rectally, intranasally), or transdermally.

44. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

45. The novel compositions disclosed herein are preferably formulated as creams or ointments. As used herein, a “cream” is a topical preparation used for application on the skin. Creams are semi-solid emulsions, which are mixtures of oil and water in which APIs (Active Pharmaceutical Ingredients) are incorporated. They are divided into two types: oil-in-water (O/W) creams which compose of small droplets of oil dispersed in a continuous water phase, and water-in-oil (W/O) creams which compose of small droplets of water dispersed in a continuous oily phase. Oil-in-water creams are user-friendly and hence cosmetically acceptable as they are less greasy and more easily washed with water. An ointment is a viscous semisolid preparation containing APIs, which are used topically on a variety of body surfaces. The vehicle of an ointment is known as ointment base. The choice of a base depends upon the clinical indication of the ointment, and the different types of ointment bases include, but are not limited to: hydrocarbon bases, e.g. hard paraffin, soft paraffin, absorption bases, e.g. wool fat, bees wax.

46. Active compounds in cream formulations are available in ionized state, whereas in case of ointments these are present in non -ionized state. Generally, cream formulations are the first choice of the formulators in design and development of topical dosage forms, as cream formulations are cosmetically elegant, and also as the active compound is available in ionized state, the drug can penetrate the skin layer fast which makes the formulation totally patient friendly.

47. Effective dosages and schedules for administering the disclosed compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

48. Following administration of a disclosed composition, such as corticosteroid in combination with a biopolymer, for treating, inhibiting, or preventing a dermatological condition, the efficacy of the composition can be assessed in various ways well known to the skilled practitioner. For instance, one of ordinary skill in the art will understand that the composition, as disclosed herein is efficacious in treating or inhibiting dermatological condition in a subject by observing that the composition reduces inflammation, induces skin repair or reduces scarring.

49. The compositions that improve wound repair and alleviate skin problems disclosed herein may be administered prophylactically to patients or subjects who are at risk for dermatological issues such as psoriasis, inflammation etc.

50. In an aspect, the compositions described herein may be used to treat wound healing.

51. In an aspect, the compositions described herein may be used to treat dermatological conditions including but not limited to acne and related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis (eczema), contact dermatitis, dermatitis herpetiformis, generalized exfoliative dermatitis, seborrheic dermatitis, rosacea, shingles, sweating disorders, vitiligo and viral skin disease

52. It is understood that the compositions disclosed herein have certain functions, such as having antinflammatory or antiinfective effects. Disclosed herein are certain structural requirements for performing the disclosed functions, and it is understood that there are a variety of structures which can perform the same function which are related to the disclosed structures, and that these structures will ultimately achieve the same result.

### C. Methods of Making the Compositions

53. The compositions disclosed herein and the compositions necessary to perform the disclosed methods can be made using any method known to those of skill in the art for that particular reagent or compound unless otherwise specifically noted.

54. Disclosed herein are methods for making compositions comprising the mixing of betamethasone component such as betamethasone proprionate or betamethasone valerate and a biopolymer in a cream base, wherein the cream base comprises a primary and a secondary emulsifier, a waxy material, a co-solvent, a preservative, an acid, a chelating agent, a buffering agent, and water.

55. The method of making the compositions described herein comprises may further comprise the incorporation of an anti-oxidant, or a humectant. In certain aspects, the methods may comprise the use of betamethasone proprionate or betamethasone valerate added in an amount between 0.001% (w/w) and 5% (w/w), between about 0.01% (w/w) and 2% (w/w), or at 1% (w/w).

56. In certain aspects, the methods described herein comprise the use of a biopolymer, wherein the biopolymer comprises chitosan. In certain aspects, the chitosan is described as being US pharmacopeia conformant with regard to its functional excipient category and selected from any grades such as long chain, medium chain and short chain, and may have a molecular weight in the range of 50 kDa to 5000 kDa. In certain aspects, the chitosan is added in an amount between 0.01% (w/w) and 2% (w/w) by weight, in an amount from 0.01% (w/w) to 1.5% (w/w), or 0.5% (w/w).

57. In an aspect, the methods described herein comprise the use of primary and secondary emulsifiers selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

58. In an aspect, the methods disclosed herein comprise a waxy material wherein the waxy material is selected from a group comprising white soft paraffin, liquid paraffin, and hard paraffin; and wherein the waxy material is added in an amount from 5% (w/w) to 30% (w/w).

59. In an aspect, the methods disclosed herein comprise the use of a co-solvent selected from a group comprising propylene glycol, hexylene glycol, polyethylene glycol-400; and wherein the co-solvent is added in an amount from about 5% (w/w) to 50% (w/w).

60. In an aspect, the methods disclosed herein comprise the use of an acid, wherein the acid is selected from a group comprising HCl, H₂SO₄, HNO₃, and lactic acid; and wherein the acid is added in an amount from about 0.005% (w/w) to 1% (w/w).

61. In an aspect, the methods disclosed herein comprise the use of a preservative, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

62. In an aspect, the buffering agent used in the methods disclosed herein is selected from the group comprising disodium hydrogen orthophosphate, sodium hydrogen orthophosphate; wherein in certain aspects, the buffering agent is added in an amount of 0.05% (w/w) to 1% (w/w).

63. In an aspect, the methods disclosed herein comprise the use of water, wherein the water is added in the range of 20% (w/w) to 75% (w/w), or 35% (w/w) to 60% (w/w).

64. In an aspect, the methods disclosed herein comprise the use of anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

65. In an aspect, the methods disclosed herein further comprise the use of a chelating agent, wherein the chelating agent is selected from the group comprising disodium EDTA; and wherein in certain aspects the chelating agent is added in an amount 0.05% (w/w) to 1% (w/w).

66. In an aspect, the methods disclosed herein further comprise the use of a humectant, wherein the humectant is selected from a group comprising glycerin, propylene glycol, sorbitol; and wherein the humectant is added in an amount of 5% (w/w) to 20% (w/w).

### D. EXAMPLES

67. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### E. Example 1

### Betamethasone Dipropionate and Chitosan Composition

TABLE 1
Betamethasone Dipropionate (0.064%) + Chitosan (0.5%) Cream

| S. No | Name of the Material | Qty (in %) |
|---|---|---|
| 1 | Betamethasone Dipropionate | 0.064 |
| 2 | Chitosan | 0.5 |
| 3 | Methylparaben | 0.2 |
| 4 | Propylparaben | 0.02 |
| 5 | Cetostearyl Alcohol | 7.2 |
| 6 | Cetomacrogol 1000 | 1.8 |
| 7 | White Soft Paraffin | 20 |
| 8 | Liquid Paraffin | 10 |
| 9 | Lactic Acid | 0.05 |
| 10 | Propylene Glycol | 11.5 |
| 11 | Purified Water | 48.61 |

TABLE 2
Betamethasone Dipropionate (0.064%) + Chitosan (0.5%) Cream

| S. No | Name of the Material | Qty (in %) |
|---|---|---|
| 1 | Betamethasone Dipropionate | 0.064 |
| 2 | Chitosan | 0.5 |
| 3 | Methylparaben | 0.2 |
| 4 | Propylparaben | 0.02 |
| 5 | Isopropyl myristate | 2.5 |
| 6 | Chlorocresol | 0.1 |
| 7 | Cetostearyl Alcohol | 7.2 |
| 8 | Cetomacrogol 1000 | 1.8 |
| 9 | White Soft Paraffin | 20 |
| 10 | Liquid Paraffin | 10 |
| 11 | Lactic Acid | 0.05 |
| 12 | Propylene Glycol | 9 |
| 13 | Purified Water | 48.51 |

68. Tables 1 and 2 provide select embodiments of the present invention comprising betamethasone dipropionate including percentage composition of individual components.

69. The compositions described in Tables 1 and 2 are made according to the process outlined in the steps below:
Step 1: Disperse Methyl Paraben and Propyl Paraben in required quantity of Purified Water at 70° C. in Vessel 1.
Step 2: Melt White soft paraffin, Cetostearyl alcohol, Cetomacrogol-1000, Light liquid paraffin and Isopropyl Myristate at 70° C. in Vessel 2 and add to the solution obtained in Step 1. Cool the combined mixture to 50° C. under continuous stirring.
Step 3: Disperse betamethasone dipropionate in Propylene Glycol and add it to the above cream base prepared in Step 2. Rinse vessel with Propylene Glycol.
Step 4: Preparation of Chitosan gel: Add Chitosan-M in the remaining Purified Water acidified with Lactic Acid in a separate vessel and add to the above base obtained in step 2 at 40° C. Cool the final cream to 25° C.-30° C. with continuous stirring.

70. The compositions claimed herein and prepared for example, according to the percentages provided in Tables 1 and 2, provide superior therapeutic efficacy as topically applied anti-inflammatory creams with chitosan. The compositions are particularly useful for the treatment of skin inflammation, dermatitis, and allergic conditions. The novel compositions described herein enable the efficient delivery of active therapeutic agents to penetrate intact skin, to improve skin regeneration and rejuvenation, as well as wound healing.

### F. Example 2

### Betamethasone Dipropionate and Chitosan API Stability

Experimental Data

71. API-Stability experiments were carried out (see Tables 3-11 below) using the compositions of the present invention. Tests were carried out to observe the physical appearance of the product, pH and assay of the API over a period of time. Tests were also carried out to assess the stability of the compositions by subjecting the compositions to stress studies such as autoclave test and oxidative degradation tests (contained approximately 5% extra API (overages). The compositions were packaged in aluminum collapsible tubes and each gram of the product contained 0.64 mg of betamethasone dipropionate (in conformance with USP) which is equivalent to 0.5 mg of betamethasone (in conformance with USP). Further, in-vitro, preclinical and clinical studies were carried out over a period of time.

TABLE 3
Description Test, Batch No. BDC-21
Measured parameter: Physical appearance
Best value of measured parameter: Homogeneous white to off white
viscous cream (C indicates compliance with acceptance limit value)
Method of measurement: Observation by naked eye

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to off | — | — | C | C | C | C | C | C | C |
| 25° C. 60% RH | white viscous | — | — | C | C | C | C | C | C | C |
| Temp. cycling | cream | — | — | — | — | — | — | — | — | — |
| Freeze thaw | | — | — | — | — | — | — | — | — | — |

TABLE 4
pH Test, Batch No. BDC-21
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.68 | 4.66 | 4.65 | 4.63 | 4.61 | — | — | — | — | — |
| 30° C. 65% RH | | — | — | 4.65 | 4.63 | 4.61 | 4.59 | 4.58 | 4.57 | 4.52 |
| 25° C. 60% RH | | — | — | 4.66 | 4.64 | 4.62 | 4.61 | 4.59 | 4.58 | 4.38 |
| Temp. cycling | | 4.55 | — | — | — | — | — | — | — | — |
| Freeze thaw | | 4.48 | — | — | — | — | — | — | — | — |

TABLE 5
Assay % Test, Batch No. BDC-21
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 24^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.82 | 104.56 | 104.05 | 103.91 | 103.87 | — | — | — | — | — |
| 30° C. 65% RH | | — | — | 104.36 | 104.07 | 103.95 | 103.88 | 103.68 | 103.21 | 102.73 |
| 25° C. 60% RH | | — | — | 104.45 | 104.27 | 103.98 | 103.77 | 103.42 | 103.18 | 102.85 |
| Temp. cycling | | 103.58 | — | — | — | — | — | — | — | |
| Freeze thaw | | 103.32 | — | — | — | — | — | — | — | |

TABLE 6
Description Test, Batch No. BDC-22
Measured parameter: Physical appearance
Best value of measured parameter: Homogeneous white to off white
viscous cream (C indicates compliance with acceptance limit value)
Method of measurement: Observation by naked eye

| | | 1^{st} | 2^{nd} | 3^{rd} | 6^{th} | 9^{th} | 12^{th} | 18^{th} | 24^{th} |
|---|---|---|---|---|---|---|---|---|---|
| Condition | Initial | Mth | Mth | Mth | Mth | Mth | Mth | Mth | Mth |
| 40° C. 75% RH | Homogeneous | C | C | C | C | — | — | — | — |
| 30° C. 65% RH | white to off | — | — | C | C | C | C | C | C |
| 25° C. 60% RH | white viscous | — | — | C | C | C | C | C | C |
| Temp. cycling | cream | C | — | — | — | — | — | — | — |
| Freeze thaw | | C | — | — | — | — | — | — | — |

TABLE 7
pH Test, Batch No. BDC-22
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.66 | 4.62 | 4.61 | 4.58 | 4.56 | — | — | — | — | — |
| 30° C. 65% RH | | — | — | 4.59 | 4.57 | 4.54 | 4.51 | 4.48 | 4.45 | 4.40 |
| 25° C. 60% RH | | — | — | 4.61 | 4.59 | 4.58 | 4.56 | 4.54 | 4.52 | 4.36 |
| Temp. cycling | | 4.51 | — | — | — | — | — | — | — | — |
| Freeze thaw | | 4.45 | — | — | — | — | — | — | — | — |

TABLE 8
Assay % Test, Batch No. BDC-22
Measured parameter: Assay (%)
Method of measurement: HPLC method
Limit of measured parameter: 90-110%

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.39 | 104.22 | 104.18 | 104.09 | 104.02 | — | — | — | — | — |
| 30° C. 65% RH | | — | — | 104.11 | 104.02 | 103.86 | 103.51 | 103.28 | 103.11 | 102.74 |
| 25° C. 60% RH | | — | — | 104.19 | 104.11 | 104.01 | 103.98 | 103.23 | 103.05 | 102.88 |
| Temp. cycling | | 103.85 | — | — | — | — | — | — | — | — |
| Freeze thaw | | 103.91 | — | — | — | — | — | — | — | — |

TABLE 9
Description Test, Batch No. BDC-23
Measured parameter: Physical appearance
Best value of measured parameter: Homogeneous white to off white
viscous cream (C indicates compliance with acceptance limit value)
Method of measurement: Observation by naked eye

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to off | — | — | C | C | C | C | C | C | C |
| 25° C. 60% RH | white viscous | — | — | C | C | C | C | C | C | C |
| Temp. cycling | cream | C | — | — | — | — | — | — | — | — |
| Freeze thaw | | C | — | — | — | — | — | — | — | — |

TABLE 10
pH Test, Batch No. BDC-23
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.69 | 4.64 | 4.62 | 4.58 | 4.55 | — | — | — | — | — |
| 30° C. 65% RH | | — | — | 4.62 | 4.59 | 4.56 | 4.53 | 4.49 | 4.45 | 4.39 |
| 25° C. 60% RH | | — | — | 4.64 | 4.61 | 4.58 | 4.55 | 4.51 | 4.49 | 4.41 |
| Temp. cycling | | 4.53 | — | — | — | — | — | — | — | — |
| Freeze thaw | | 4.43 | — | — | — | — | — | — | — | — |

TABLE 11
Assay % Test, Batch No. BDC-23
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.21 | 104.05 | 103.98 | 103.92 | 103.89 | — | — | — | — | — |
| 30° C. 65% RH | | — | — | 103.98 | 103.91 | 103.87 | 103.85 | 103.81 | 103.77 | 102.45 |
| 25° C. 60% RH | | — | — | 104.03 | 103.96 | 103.91 | 103.89 | 103.86 | 103.81 | 102.83 |
| Temp. cycling | | 103.85 | — | — | — | — | — | — | — | — |
| Freeze thaw | | 103.71 | — | — | — | — | — | — | — | — |

TABLE 12

| Sample Name | Mfg Date | Expiry Date |
|---|---|---|
| Present Invention | April 2011 | March 2014 |
| (Betamethasone Dipropionate Cream) | | |

TABLE 13
Autoclave Analysis (%) Test
Measured parameter: Assay (%)
Method of measurement: HPLC method

| | | Analysis-I (%) | | | Analysis-II (%) | | | Average Drop of |
|---|---|---|---|---|---|---|---|---|
| | Name of the | | After | | | After | | Analysis-I and |
| S. No | Products | Initial | Autoclave | Drop | Initial | Autoclave | Drop | Analysis -II (%) |
| 1 | Present Invention | 104.82 | 103.18 | 1.64 | 104.82 | 103.21 | 1.61 | 1.63 |

TABLE 14
Oxidative degradation Analysis (%) Test
Measured parameter: Assay (%)
Method of measurement: HPLC method

| | | Analysis (%) | | |
|---|---|---|---|---|
| S. No. | Name of the products | Initial | After Oxidation | Degradation |
| 1 | Present Invention | 104.82 | 103.09 | 1.73 |

72. Summary: From the above data, it is evident that the composition of the present invention is stable at ambient conditions, at elevated temperatures and humid conditions of storage. Also the autoclave studies and oxidative degradation studies further confirm the stability of the product. This is a significant advantage over currently available betamethasome creams. The stability of the product is further ascertained by the shelf-life prediction of the formulation using Arrhenius plot of degradation employing Nova-LIMS software.

### G. Example 3

### Application of Betamethasone Dipropionate and Chitosan Compositions

Method of Application

73. In an embodiment, the compositions (creams) as disclosed herein are applied after thorough cleansing and drying the affected skin area. The compositions are applied in an amount sufficient to cover the affected skin and surrounding area. The compositions may be applied 1-10 times a day, 2-3 times a day, 1-4 times day, or as necessary depending upon the skin conditions for a full treatment period, even though symptoms may have improved. A full treatment period may be determined by one skilled in the art, such as a health care provider, including but not limited to a physician. In an embodiment, the betamethasone dipropionate and chitosan composition of the present invention may be applied once to twice daily to the affected area: for some subjects, adequate maintenance may be achieved with less frequent application.

Studies

74. Experimental studies were conducted using the presently described compositions (creams) in the laboratory as well as using suitable animal models, i.e. pig ear skin. The aspects tested included—diffusion study, film forming, skin erythema, and clinical trial. These aspects together demonstrate that the present invention is effective in wound healing.

Diffusion Study:

75. A diffusion study comprising the composition of the present invention (betamethasone diproprionate with chitosan) and the reference product was determined using a pig ear skin model. The results indicated that the present invention and the reference product demonstrated poor diffusion and hence may show negligible absorption when tested in in vivo in human volunteers. Accordingly, it is concluded that the formulation can be applied for the treatment of local inflammation with an advantage that the drug will be localized at the site of inflammation thereby providing better therapeutic effect. The release rate of betamethasone dipropionate cream of the present invention was found to be 4.6×10⁻⁵/hr when compared with reference product shown 2.3×10⁻⁵/hr at the end of 8 hours.

Film Forming Properties:

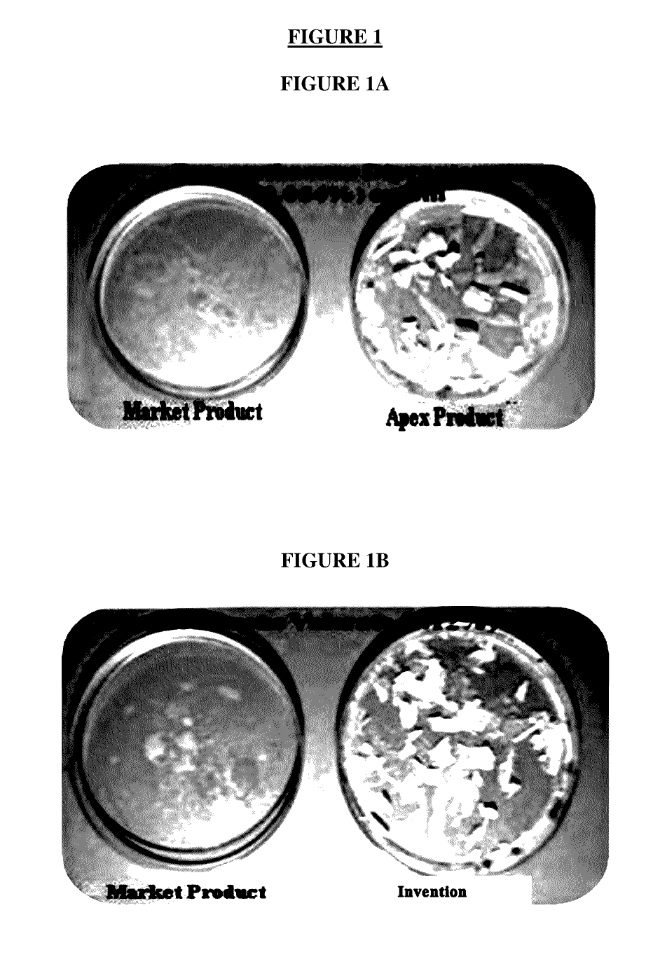

76. It is evident from FIG. 1 that chitosan does not lose its film forming property in the presence of the excipients used for cream preparations in the present invention. Indeed, chitosan doesn't change its film forming property even in the presently described novel compositions and this ensures that a thin film is formed when cream formulation is applied over the skin. The film formation ensures the moisturizing and soothing effect of the cream and also the even distribution of the active component is ensured when applied over skin. This property particularly valuable when compared to the existing marketed cream formulations.

Skin Erythema Study:

77. A skin inflammatory Study was carried out in Wistar rat divided in to three groups i.e a control group, a group treated with the cream of the present invention, and a group treated with reference product. On application of Croton oil in ear of rats, it was observed that it produced 70% edema in control group. From the study it was concluded that both the formulations betamethasone dipropionate cream of invention and the reference product were effective in reduction of croton oil induced edema. However, it was surprisingly found that the betamethasone dipropionate cream of invention produced substantially superior results (78.11%) in reduction in croton oil induced edema compared with the reference product (65.18%).

TABLE 15
Effect of Betamethasone Dipropionate Cream and Reference
product on croton oil induced skin edema

| | Edema % | |
|---|---|---|
| Group | (Mean ± SEM) | Protection % |
| Control | 70.84 ± 3.41 | — |
| Betamethasone Dipropionate | 15.50 ± 2.54 | 78.11 |
| Cream of invention | | |
| Reference product | 24.66 ± 2.08 | 65.18 |

Acute Dermal Irritation Study

78. Skin irritation may be the result of numerous causes, including but not limited to topical exposure to chemicals, drugs, and other toxins or harmful activities such as abrasions or laceration. Depending on the severity of the irritation, and depending on the cause of the irritation, skin damage may be reversible. In designing the appropriate treatment, harmful products may be categorized as irritants or corrosive. The present experimental study was performed to assess the possible hazard likely to arise from exposure of topical formulations to the human skin. Thus a primary skin irritation study was carried out for the composition claimed herein, a newly formulated dermal cream, betamethasone dipropionate cream comprising chitosan to determine its irritant response to the skin after single exposure. From the experimental study it was concluded that the formulation of betamethasone dipropionate cream (invention) score for the primary skin irritation index was 0. Hence, the betamethasone dipropionate cream (invention) was non-irritant and dermal-friendly.

Study Design:

79. Totally three male Oryctolagus cuniculus (Rabbit)-New Zealand white (2114.78-2396.31 g) were used. Quantity of 0.5 g of each Betamethasone Dipropionate Cream and Betamil Cream was weighed and applied as such. Approximately 24 hr before the test/ reference substance application, the hairs were closely clipped on both side of dorso-lateral surface of each animal so as to have two sites on each side (A,B in left side and C, D in right side) with an area of 6 cm2 per site. Care was take to avoid abrasion on the skin and animal with intact skin alone were used for the experiment. Test substances—Betamethasone Dipropionate Cream (Apex) and reference substance—Betamil Cream (Merck) were applied to the clipped sites A & D respectively. The other two sites B& C in left and right side were treated as control sites. All sites were covered with a non-oclclusive absorbent gauze patch and was held in place with non-irritating tape. The patch was then wrapped with semi-occlusive bandage for the duratuion of the exposure period. At the end of exposure period, the residual test/reference substance was removed, using lukewarm water without altering the existing response or the integirity of the epidermis. Initially the experiment was performed using one animal with application of test and reference substance. No dermal reactions were observed at both test site and reference site at the end of 4 hrs exposure period. The conformation test was carried out with two additional animals with an exposure period of 4 hrs in order to confirm the non-irritant nature of both test and reference substances. All animals were observed for a period of 1, 24, 48 and 72 hr following the removal of gauze patch.

Results:

80. Body weight of each animal was recorded prior to the application of test/reference substance (Table: A)

TABLE A
Individual Body Weight Data

| Rabbit No. | Sex | Body weight (g) |
|---|---|---|
| 1 | Male | 2291.14 |
| 2 | Male | 2396.31 |
| 3 | Male | 2114.78 |

81. In the initial and confirmatory tests, none of the animals showed any skin reactions at 0, 1, 24, 48 and 72 hrs after the patch removal in control and treated sites (both test and reference). In the initial and confirmatory tests, none of the animals showed any skin reactions at 0, 1, 24, 48 and 72 hrs after the patch removal in control and treated sites (both test and reference). None of the animals exhibited clinical signs of toxicity or mortality. Based on the observation the Primary Skin Irritation Index of Betamethasone Dipropionate Cream (Apex) and Betamil Cream (Merck) were calculated as 0.

Bleeding Time Study

82. A bleeding time study was performed in both groups of animals—untreated control group and the test group treated with the product of present invention. Statistically significant decrease in the bleeding time in treated group animals was observed when compared with that of the control group animals. The mean percent reduction in bleeding time of 30.14% was observed for the treated group animals with the product of present invention.

Study Design:

83. Totally six Male and Female Oryctolagus cuniculus (Rabbit)—New Zealand white Rabbits were used. Approximately 24 hr prior to initiation of the experiment, hair on both ear lobes of all experimental rabbits was removed by using depilatory cream. On the day of application, ear lobes were rubbed gently with cotton dipped in lukewarm water for vein dilation to enhance the visibility of vein. Then the ear lobes were wiped with 70% alcohol. Approximately 5 mm full thickness incision was made in the marginal ear vein with a sterile lancet. Immediately when blood started to flow, the stopwatch was switched on and the duration of the bleeding was measured by the absorption of blood onto the filter paper at every 15 second interval without disturbing the incision site. Incision in right ear lobe was left as such, whereas the incision in the left ear lobe was applied immediately with 350 mg of the test item. The time taken for bleeding to stop in the right and left lobe was considered as control and treatment bleeding time respectively.

Results:

84. The body weight of all animals was recorded on the treatment day is tabulated in (Table B). None of the animals showed any clinical signs and mortality during the experimental period.

85. Bleeding time observed in both control side and treated side of the same animals were shown in (Table C). Bleeding time of all animals showed significant difference between the control and treatment sides (P<0.0096). No gender-specific significant difference was observed between the treatment and control sides.

TABLE B
Body Weight in Grams (Individual and Mean)

| Animal No | Sex | Body Weight |
|---|---|---|
| 1 | M | 2498.28 |
| 2 | M | 2418.22 |
| 3 | M | 2367.92 |
| | Mean | 2428.14 |
| | S.D. | 53.68 |
| 4 | F | 2348.39 |
| 5 | F | 2296.33 |
| 6 | F | 2245.28 |
| | Mean | 2296.67 |
| | S.D. | 42.10 |

M: Male;
F: Female;
S.D: Standard Deviation

TABLE C
Blood Bleeding Time (in Seconds)

| Animal No | Sex | Control | Mean | Treatment | Mean |
|---|---|---|---|---|---|
| 1 | M | 125.00 | 123.67 | 81.00 | 81.00 |
| 2 | M | 126.00 | | 87.00 | |
| 3 | M | 120.00 | | 75.00 | |
| 4 | F | 178.00 | 172.67 | 120.00 | 126.00 |
| 5 | F | 130.00 | | 130.00 | |
| 6 | F | 210.00 | | 128.00 | |
| Mean | | 148.17 | | 103.50 | |
| S.D. | | 37.05 | | 25.16 | |

M: Male;
F: Female;
S.D: Standard Deviation Values are statistically different (P < 0.0096)

### CONCLUSIONS

86. From the above experimental results, it may be concluded that the test substance Betamethasone Dipropionate USP 0.064% w/w cream was effective in reducing the bleeding time (30.14%) in New Zealand White rabbits following topical application.

Clinical Study

87. The study was a randomized, double blind, controlled clinical trial in patients with skin infections (eczema, dermatitis, allergies and rash), inflammatory and pruritic manifestations of corticosteroid responsive dermatosis using betamethasone dipropionate cream of the invention and market sample.
a. Visual Analogue Scale (VAS) score clearly indicates that severity of wound was lesser in test group (betamethasone dipropionate cream of invention).
b. Global Score Index (GSI) indicates that severity of eczematous dermatitis was lesser in betamethasone dipropionate cream of invention.
c. Summary statistics of Patient's compliance confirmed that 40% of study population has achieved score zero i.e. absence of signs of itching or indication of pain from the group, that received betamethasone dipropionate Cream of invention, but only 0% of study population achieved with reference product.
d. Physician Global Evaluation (PGE) score shows that 70% of study population from group that received betamethasone dipropionate cream of invention achieved good and excellent results but only 0% achieved good and excellent results with reference product.
e. Based on the statistical results obtained from this study it is concluded that, betamethasone dipropionate cream of invention than the reference product, shown to be clinically equivalent in terms of anti-inflammatory activity.

Results and Discussion

88. The therapeutic impact, as observed from the animal and human volunteers testing, and addition of chitosan to betamethasone dipropionate, an anti inflammatory agent, is shown in the following table (Table 16) by considering various aspects of therapeutic cure of skin inflammatory condition.

TABLE 16

| Therapeutic aspect | | Product of present invention |
|---|---|---|
| 1. | Diffusion study | The rate of release of Betamethasone Dipropionate |
| | | Cream of invention was found to be 4.6 × 10⁻⁵/hr |
| | | when compared with reference product shown 2.3 × |
| | | 10⁻⁵/hr at the end of 8 hrs. |
| 2. | Skin Erythema | Betamethasone Dipropionate Cream of invention |
| | study | was shown more effectiveness (78.11%) in pre- |
| | | venting the development of croton oil induced |
| | | edema than the reference product shown (65.18%). |
| 3. | Acute dermal | Betamethasone Dipropionate Cream of invention |
| | irritation study | is non-irritant. |
| 4. | Bleeding Time | Betamethasone Dipropionate Cream of invention |
| | study | has 30.14% mean percent reduction in bleeding |
| | | time in animals when compared to control group |
| 5. | Film forming | Yes (see FIG. 1) |
| | property | |
| 6. | Clinical trial | Betamethasone Dipropionate Cream of invention |
| | | had scored better than reference product based |
| | | on the various parameters such as VAS score, |
| | | GSI score, PGE score and Patient's compliance. |

Results and Discussions

89. As described herein, the novel compositions of the present invention, comprising chitosan and betamethasone dipropionate, are superior in therapeutic efficacy compared to currently available comparative medicaments. Though not wishing to be bound by the following theory, it is expected that the unique and innovative combination and selection of specific excipients results in achieving the superior results demonstrated herein.

90. The therapeutic impact, as observed from the animal testing and on human volunteers is a result of the novel compositions disclosed herein, wherein said compositions comprise chitosan and betamethasone dipropionate.

91. Though not wishing to be bound by the following theory, it is believed that the film forming ability of the chitosan incorporated in the cream allows better access of the anti-inflammatory agent to the inflamed area and results in better functioning of these API, importantly resulting in improved healing.

92. It is evident from the foregoing discussion that the present invention offers the following advantages and unique aspects over the currently available dermaceutical compositions for anti-inflammatory effect and pruritic manifestations of corticosteroid responsive dermatoses of the skin.
The compositions of the present invention include a skin-compatible biopolymer in the form of chitosan which enables enhanced therapeutic outcomes. Such enhanced therapeutic outcomes include, but are not limited to, faster relief from skin infection and inflammation.
The compositions of the present invention uniquely incorporate a biopolymer without compromising the stability of the cream matrix and without adversely affecting the functioning of known active pharmaceutical ingredient. The resulting compositions unexpectedly achieve such results through a careful selection of functional excipients to bypass undesirable aspects of physio-chemical compatibility/stability and bio-release.
The compositions of the present invention provide an integrated unit-dose or a single-dose therapy hitherto unavailable in prescription dermaceutical formulations.
The novel compositions of the present invention are adequately stable/efficacious at ambient conditions and do not need special temperature control during transportation/storage.

### H. Example 4

### Betamethasone Valerate and Chitosan Composition

TABLE 17
Betamethasone Valerate (0.12%) + Chitosan (0.5%)Cream

| S. No | Component | Qty (in %) |
|---|---|---|
| 1 | Betamethasone Valerate | 0.12 |
| 2 | Chitosan | 0.5 |
| 3 | Methylparaben | 0.2 |
| 4 | Propylparaben | 0.02 |
| 5 | Cetostearyl Alcohol | 7.2 |
| 6 | Cetomacrogol 1000 | 1.8 |
| 7 | White Soft Paraffin | 20 |
| 8 | Liquid Paraffin | 10 |
| 9 | Lactic Acid | 0.05 |
| 10 | Propylene Glycol | 11.5 |
| 11 | Purified Water | 48.61 |

TABLE 18
Betamethasone Valerate (0.12%) + Chitosan (0.5%) Cream

| S. No | Name of the Material | Qty (in %) |
|---|---|---|
| 1 | Betamethasone Valerate | 0.12 |
| 2 | Chitosan | 0.5 |
| 3 | Methylparaben | 0.2 |
| 4 | Propylparaben | 0.02 |
| 5 | Isopropyl myristate | 2.5 |
| 6 | Chlorocresol | 0.1 |
| 7 | Cetostearyl Alcohol | 7.2 |
| 8 | Cetomacrogol 1000 | 1.8 |
| 9 | White Soft Paraffin | 20 |
| 10 | Liquid Paraffin | 10 |
| 11 | Lactic Acid | 0.05 |
| 12 | Propylene Glycol | 9 |
| 13 | Purified Water | 48.51 |

93. Tables 17 and 18 provide two embodiments of the present invention including percentage composition of individual components.

94. The composition described in the above Tables are made according to the process outlined in the steps below:
Step 1: Disperse Methyl Paraben and Propyl Paraben in required quantity of Purified Water at 70° C. in Vessel 1.
Step 2: Melt White soft paraffin, Cetostearyl alcohol, Cetomacrogol-1000, Light liquid paraffin at 70° C. in Vessel 2 and add to the solution obtained in Step 1. Cool the combined mixture to 50° C. under continuous stirring.
Step 3: Disperse Betamethasone Valerate in Propylene Glycol and add it to the above cream base prepared in Step 2.
Step 4: Preparation of Chitosan gel: Add Chitosan-M in the remaining Purified Water acidified with Lactic Acid in a separate vessel and add to the above base obtained in step 2 at 40° C. Cool the final cream to 25° C.-30° C. with continuous stirring.

95. The compositions claimed herein and prepared for example, according to the percentages provided in Tables 17 and 18, provide superior therapeutic efficacy as topically applied anti-inflammatory creams with chitosan. The compositions are particularly useful for the treatment of skin inflammation, dermatitis, and allergic conditions. The novel compositions described herein enable the efficient delivery of active therapeutic agents to penetrate intact skin, to improve skin regeneration and rejuvenation, as well as wound healing.

### I. Example 5

### Betamethasone Dipropionate and Chitosan API Stability

Experimental Data

96. API-Stability experiments were carried out (see Tables 19-30 below) using the compositions of the present invention. Tests were carried out to observe (or measure as appropriate) the physical appearance of the product, pH and assay of the API over a period of time. Tests were also carried out to assess the stability of the compositions by subjecting the product to stress studies such as autoclave test and oxidative degradation tests (containing approximately 5% extra API (overages). The compositions were packaged in aluminium collapsible tubes and each gram of the product contained 1.2 mg of betamethasone valerate (in conformance with USP) which is equivalent to 1 mg of betamethasone (in conformance with USP). Further, in-vitro, preclinical and clinical studies were carried out over a period of time.

TABLE 19
Description test, Batch No. BVC-27
Measured parameter: Physical appearance
Method of measurement: Observation by naked eye
Best value of measured parameter: Homogeneous white to off white
viscous cream (C indicates that the results comply with the initial state)

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to off | — | — | C | C | C | C | C | C | C |
| 25° C. 60% RH | white viscous | — | — | C | C | C | C | C | C | C |
| Temp. cycling | cream | C | — | — | — | — | — | — | — | — |
| Freeze thaw | | C | — | — | — | — | — | — | — | |

TABLE 20
pH test, Batch No. BVC-27
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.56 | 4.49 | 4.46 | 4.44 | 4.42 | — | — | — | — | — |
| 30° C. 65% RH | | — | — | 4.45 | 4.40 | 4.38 | 4.35 | 4.33 | 4.31 | 4.18 |
| 25° C. 60% RH | | — | — | 4.48 | 4.45 | 4.41 | 4.39 | 4.38 | 4.36 | 4.16 |
| Temp. cycling | | 4.48 | — | — | — | — | — | — | — | — |
| Freeze thaw | | 4.46 | — | — | — | — | — | — | — | — |

TABLE 21
Assay % test, Batch No. BVC-27
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.61 | 104.42 | 104.37 | 104.32 | 104.26 | — | — | — | — | — |
| 30° C. 65% RH | | — | — | 104.40 | 104.34 | 104.31 | 104.26 | 104.12 | 104.01 | 103.26 |
| 25° C. 60% RH | | — | — | 104.47 | 104.42 | 104.36 | 104.28 | 104.23 | 104.17 | 103.04 |
| Temp. cycling | | 104.36 | — | — | — | — | — | — | — | — |
| Freeze thaw | | 104.32 | — | — | — | — | — | — | — | — |

TABLE 22
Description test, Batch No. BVC-28
Measured parameter: Physical appearance
Best value of measured parameter: Homogeneous white to off white
viscous cream (C indicates compliance with the initial state)
Method of measurement: Observation by naked eye

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to off | — | — | C | C | C | C | C | C | C |
| 25° C. 60% RH | white viscous | — | — | C | C | C | C | C | C | C |
| Temp. cycling | cream | C | — | — | — | — | — | — | — | — |
| Freeze thaw | | C | — | — | — | — | — | — | — | — |

TABLE 23
pH test, Batch No. BVC-28
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.72 | 4.68 | 4.65 | 4.61 | 4.56 | — | — | — | — | — |
| 30° C. 65% RH | | — | — | 4.68 | 4.65 | 4.63 | 4.62 | 4.58 | 4.56 | 4.47 |
| 25° C. 60% RH | | — | — | 4.70 | 4.67 | 4.65 | 4.63 | 4.63 | 4.60 | 4.53 |
| Temp. cycling | | 4.69 | — | — | — | — | — | — | — | — |
| Freeze thaw | | 4.70 | — | — | — | — | — | — | — | — |

TABLE 24
Assay % test, Batch No. BVC-28
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.20 | 104.13 | 104.04 | 103.95 | 103.89 | — | — | — | — | — |
| 30° C. 65% RH | | — | — | 104.04 | 103.95 | 103.89 | 103.81 | 103.74 | 103.68 | 103.24 |
| 25° C. 60% RH | | — | — | 104.07 | 104.01 | 103.96 | 103.90 | 103.82 | 103.75 | 102.78 |
| Temp. cycling | | 103.95 | — | — | — | — | — | — | — | — |
| Freeze thaw | | 103.03 | — | — | — | — | — | — | — | — |

TABLE 25
Description test, Batch No. BVC-29
Measured parameter: Physical appearance
Best value of measured parameter: Homogeneous white to off white
viscous cream (C indicated compliance with initial value)
Method of measurement: Observation by naked eye

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | Homogeneous | C | C | C | C | — | — | — | — | — |
| 30° C. 65% RH | white to off | — | — | C | C | C | C | C | C | C |
| 25° C. 60% RH | white viscous | — | — | C | C | C | C | C | C | C |
| Temp. cycling | cream | C | — | — | — | — | — | — | — | — |
| Freeze thaw | | C | — | — | — | — | — | — | — | — |

TABLE 26
pH test, Batch No. BVC-29
Measured parameter: pH
Limit of measured parameter: 4.0-5.5
Method of measurement: Digital pH meter

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 4.43 | 4.40 | 4.38 | 4.37 | 4.35 | — | — | — | — | — |
| 30° C. 65% RH | | — | — | 4.39 | 4.37 | 4.35 | 4.32 | 4.30 | 4.28 | 4.23 |
| 25° C. 60% RH | | — | — | 4.40 | 4.38 | 4.36 | 4.35 | 4.33 | 4.32 | 4.29 |
| Temp. cycling | | 4.40 | — | — | — | — | — | — | — | — |
| Freeze thaw | | 4.42 | — | — | — | — | — | — | — | — |

TABLE 27
Assay % Test, Batch No. BVC-29
Measured parameter: Assay (%)
Limit of measured parameter: 90-110%
Method of measurement: HPLC method

| | | 1^{st} | 2^{nd} | 3^{rd} | 6^{th} | 9^{th} | 12^{th} | 18^{th} | 24^{th} | 36^{th} |
|---|---|---|---|---|---|---|---|---|---|---|
| Condition | Initial | Mth | Mth | Mth | Mth | Mth | Mth | Mth | Mth | Mth |
| 40° C. 75% RH | 104.34 | 104.21 | 104.16 | 104.08 | 104.02 | — | — | — | — | — |
| 30° C. 65% RH | | — | — | 104.18 | 104.11 | 104.05 | 104.00 | 103.93 | 103.87 | 103.51 |
| 25° C. 60% RH | | — | — | 104.20 | 104.14 | 104.08 | 104.03 | 103.95 | 103.91 | 102.66 |
| Temp. cycling | | 104.18 | — | — | — | — | — | — | — | — |
| Freeze thaw | | 104.20 | — | — | — | — | — | — | — | — |

TABLE 28

| Sample Name | Mfg Date | Expiry Date |
|---|---|---|
| Present Invention | April 2011 | March 2014 |
| (Betamethasone Valerate Cream) | | |

TABLE 29
Autoclave analysis (%) test
Measured parameter: Assay (%)
Method of measurement: HPLC method

| | | Analysis-I (%) | | | Analysis-II (%) | | | Average Drop of |
|---|---|---|---|---|---|---|---|---|
| | Name of the | | After | | | After | | Analysis-I and |
| S. No | Products | Initial | Autoclave | Drop | Initial | Autoclave | Drop | Analysis - II (%) |
| 1 | Present Invention | 104.61 | 103.34 | 1.27 | 104.61 | 103.11 | 1.50 | 1.39 |

TABLE 30
Oxidative degradation analysis (%) test
Measured parameter: Assay (%)
Method of measurement: HPLC method

| | Name of the products | Analysis (%) | | |
|---|---|---|---|---|
| S. No. | and Details | Initial | After Oxidation | Drop in % |
| 01 | Present Invention | 104.61 | 103.28 | 1.33 |

97. Summary: From the above data, it is evident that the composition of the present invention is stable at ambient conditions, at elevated temperatures and humid conditions of storage. Also the autoclave studies and oxidative degradation studies further confirm the stability of the product. This is a significant advantage over currently available creams. The stability of the product is further ascertained by the shelf-life prediction of the formulation using Arrhenius plot of degradation employing Nova-LIMS software.

### J. Example 6

### Application of Betamethasone Dipropionate and Chitosan Compositions

Method of Application

98. In an embodiment, the compositions (creams) as disclosed herein are applied after thorough cleansing and drying the affected skin area. The compositions are applied in an amount sufficient to cover the affected skin and surrounding area. The compositions may be applied 1-10 times a day, 2-3 times a day, 1-4 times day, or as necessary depending upon the skin conditions for a full treatment period, even though symptoms may have improved. A full treatment period may be determined by one skilled in the art, such as a health care provider, including but not limited to a physician.

Studies

99. Experimental studies were conducted using the presently described compositions (creams) in the laboratory as well as using suitable animal models and in human volunteers. The aspects tested included—diffusion study, film forming, skin erythema study, and clinical efficacy. These aspects together demonstrate that the present invention is effective in wound healing.

Diffusion Study

100. Compositions of the present invention comprising betamethasone valerate were assessed in diffusion studies using pig ear skin. The results indicate the betamethasone valerate cream of the present invention and reference product showed poor diffusion and hence negligible absorption when tested in in-vivo in human volunteers. It is concluded therefore that both the formulation can be applied for the treatment of local inflammation with an advantage that the drug will be localized at the site of inflammation there by providing improved therapeutic effect. The release rate of betamethasone valerate cream of the invention was found to be 9.21×10⁻⁵/hr when compared with reference product shown 6.91×10⁻⁵/hr at the end of 9 hrs.

Film Forming Properties:

101. It is evident from FIG. 1 that Chitosan does not lose its film forming property in the presence of the excipients used for cream preparations in the present invention. Indeed, chitosan doesn't change its film forming property even in the presently described novel compositions and this ensures that a thin film is formed when cream formulation is applied over the skin. The film formation ensures the moisturizing and soothing effect of the cream and also the even distribution of the active component is ensured when applied over skin. This property particularly valuable when compared to the existing marketed cream formulations.

Skin Erythema Study:

102. A skin inflammatory study was carried out in wistar rats divided into three group: control group, group treated with product of the present invention, and group treated with reference product. Application of croton oil in ear of rats has produced 70% edema in control group. The formulations of betamethasone valerate cream of the invention and reference product were effective in reduction croton oil induced edema. Betamethasone valerate cream of the invention (67.15%) had shown the highest percentage of reduction in croton oil induced edema than reference product (66.79%).

Study Design:

103. Croton Oil Ear Edema in Rats

104. The above study was carried out on arachidonic acid mice model (40) and Croton oil ear edema model of rat (40). The study was conducted on four groups of rats (10 in each group) of either sex weighing 150-200 g. The irritant croton oil was prepared by dissolving 4 parts of croton oil, 10 parts of ethanol, 20 parts of pyridine, and 66 parts of ethyl ether. The test compounds were dissolved (5 mg/ml strength) in the croton oil. The control and the test animals will receive the drug in following manner under ether anesthesia.
Group 1—0.02 ml of croton oil solution, applied on either side of the right ear.
Group 2—0.02 ml of croton oil solution containing dissolved Betamethasone Valerate Cream A—contains 0.25% chitosan (formulation of the present invention) 5 mg/ml.
Group 3—0.02 ml of croton oil solution containing dissolved Betamethasone Valerate Cream B—contains 0.50% chitosan (formulation of the present invention) 5 mg/ml.
Group 4—0.02 ml of croton oil solution containing dissolved BETNOVATE Cream—GSK 5 mg/ml.

105. Four hours after the application the animals were sacrificed under anaesthesia. Both ears are removed and discs of 8 mm diameter are punched. The discs are weighed immediately and the difference in weight between the treated and the untreated ear is indicating the degree of inflammatory edema.

106. Topical Application of Arachidonic Acid
The study was conducted on four groups of mice (10 in each group) of either sex weighing 25 g. The control and the test animals will receive the drug in following manner.
Group 1—1 mg of arachidonic acid on right ear topically, vehicle to left ear
Group 2—Betamethasone Valerate Cream A—contains 0.25% chitosan (formulation of the present invention) 5 mg/ml in acetone 30 min prior to arachidonic acid on right ear.
Group 3—Betamethasone Valerate Cream B—contains 0.50% chitosan (formulation of the present invention) 5 mg/ml in acetone 30 min prior to arachidonic acid on right ear.
Group 4—BETNOVATE Cream (GSK)—5 mg/ml in acetone 30 min prior to arachidonic acid on right ear.
107. One hour after the application the animals are sacrificed under anesthesia. Both ears are removed and discs of 8 mm diameter are punched. The discs are weighed immediately and the difference in weight between the treated and the untreated ear is indicating the degree of inflammatory edema.

108. Result:

1) Croton Oil Model

| | Edema % | |
|---|---|---|
| Group | (mean ± SEM) | P value |
| GROUP 1 | 71.80 ± 7.20 | |
| GROUP 2 | 25.55 ± 5.44 | 0.000 |
| GROUP 3 | 63.39 ± 16.01 | 1.000 |
| GROUP 4 | 43.05 ± 9.40 | 0.995 |

2) Arachidonic Model

| | Edema % | |
|---|---|---|
| Group | (mean ± SEM) | P value |
| GROUP 1 | 27.16 ± 2.00 | |
| GROUP 2 | 36.23 ± 9.69 | 1.000 |
| GROUP 3 | 28.25 ± 9.14 | 1.000 |
| GROUP 4 | 47.86 ± 9.86 | 1.000 |

109. Conclusion:

110. In vivo animal model studies under controlled laboratory condition have extensively been used by medical researsh scientists as highly dependable indicator of externally applied derma products safety, tolerability irritancy and efficacy of such studies have received widespread acceptance by regulatory agencies and dermatologists all over the world.

111. In vivo animal models also provide a highly standardized platform for meaningful comparison of derma products in current medical practice based on the above promise the result of the animal studies have significance and importance in treating occupational and topical skin disorders caused by irritants and allergens in patients. The highly statistically significant and important results were seen with Betamethasone Valerate cream A, as described herein in terms of reduction in ear edema in comparison to control group in croton oil inflammatory model but not arachidonic acid model. Neither betamethasone valerate cream B (present invention) nor Betnovate cream of GSK showed any significant result in both the models. The Betamethasone Valerate cream of the present invention would be clinically useful in treating patients with allergen/irritant included skin disorders.

TABLE 31
Effect of Betamethasone Valerate Cream and reference
product on croton oil induced skin edema

| | Edema % | |
|---|---|---|
| Group | (Mean ± SEM) | Protection % |
| Control | 70.84 ± 3.41 | — |
| Betamethasone Valerate | 23.27 ± 5.17 | 67.15 |
| Cream of the invention | | |
| Reference product | 23.52 ± 4.02 | 66.79 |

Clinical Trial:

112. A randomized, parallel group, double blinded active controlled clinical trial comparing efficacy of betamethasone valerate 0.12% cream of the invention with the reference product:
a. Visual Analogue Scale (VAS) score clearly indicated that severity of wound was decreased following the use of the betamethasone valerate cream of the present invention compared to a reference product.
b. Global Score Index (GSI) indicates that severity of eczematous dermatitis was decreased following the use of the betamethasone valerate cream of the present invention compared to a reference product.
c. Summary statistics of Patient's Compliance confirmed that 70% of study population has achieved score zero i.e. absence of signs of itching or indication of pain from the group that received betamethasone valerate cream of the invention, but only 0% of study population achieved with reference product.
d. Physician Global Evaluation (PGE) score shows that 40% population from group, that received betamethasone valerate cream of the invention achieved good and excellent results but only 0% achieved good and excellent results with reference product.

113. Based on the statistical results obtained from this study it is concluded that, betamethasone valerate 0.12% w/w cream of the invention than the reference product, shown to be clinically equivalent in terms of anti-inflammatory activity.

Acute Dermal Irritation Study

114. Skin irritation may be the result of numerous causes, including but not limited to topical exposure to chemicals, drugs, and other toxins or harmful activities such as abrasions or laceration. Depending on the severity of the irritation, and depending on the cause of the irritation, skin damage may be reversible. In designing the appropriate treatment, harmful products may be categorized as irritants or corrosive. The present experimental study was performed to assess the possible hazard likely to arise from exposure of topical formulations to the human skin. Thus a primary skin irritation study was carried out for the composition claimed herein, a newly formulated dermal cream, betamethasone valerate cream comprising chitosan to determine its irritant response to the skin after single exposure. From the experimental study it was concluded that the formulation of betamethasone valerate cream (invention) score for the primary skin irritation index was 0. Hence, the betamethasone valerate cream (invention) was non-irritant and dermal-friendly.

Study Design:

115. Totally three male Oryctolagus cuniculus (Rabbit)—New Zealand white (2169.19-2314.22 g) were used. Quantity of 0.5 g of each Betamethasone Valerate Cream and Betnovate skin Cream was weighed and applied as such. Approximately 24 hr before the test/ reference substance application, the hairs were closely clipped on both side of dorso-lateral surface of each animal so as to have two sites on each side (A, B in left side and C, D in right side) with an area of 6 cm⁻² per site. Care was take to avoid abrasion on the skin and animal with intact skin alone were used for the experiment. Test substances- Betamethasone Valerate Cream (present invention) and reference substance—Betnovate skin Cream (GSK) were applied to the clipped sites A & D respectively. The other two sites B & C in left and right side were treated as control sites. All sites were covered with a non-occlusive absorbent gauze patch and was held in place with non-irritating tape. The patch was then wrapped with semi-occlusive bandage for the duration of the exposure period. At the end of exposure period, the residual test/reference substance was removed, using lukewarm water without altering the existing response or the integrity of the epidermis. Initially the experiment was performed using one animal with application of test and reference substance. No dermal reactions were observed at both test site and reference site at the end of 4 hrs exposure period. The conformation test was carried out with two additional animals with an exposure period of 4 hrs in order to confirm the non-irritant nature of both test and reference substances. All animals were observed for a period of 1, 24, 48 and 72 hr following the removal of gauze patch.

116. Result:

117. Body Weight of each Animal was Recorded Prior to the Application of Test/Reference Substance:

Individual Body Weight Data

| Rabbit No. | Sex | Body weight (g) |
|---|---|---|
| 1 | Male | 2278.25 |
| 2 | Male | 2169.19 |
| 3 | Male | 2314.22 |

118. In the initial and confirmatory tests, none of the animals showed any skin reactions at 0, 1, 24, 48 and 72 hrs after the patch removal in control and treated sites (both test and reference). In the initial and confirmatory tests, none of the animals showed any skin reactions at 0, 1, 24, 48 and 72 hrs after the patch removal in control and treated sites (both test and reference). None of the animals exhibited clinical signs of toxicity or mortality. Based on the observation the Primary Skin Irritation Index of Betamethasone Valerate Cream of the present invention and Betnovate Cream of GSK were calculated as 0.

Results and Discussions

119. As described herein, the novel compositions of the present invention, comprising chitosan and betamethasone valerate, are superior in therapeutic efficacy compared to currently available comparative medicaments. Though not wishing to be bound by the following theory, it is expected that the unique and innovative combination and selection of specific excipients results in achieving the superior results demonstrated herein.

120. The therapeutic impact, as observed from the animal testing and on human volunteers as a result of the novel compositions disclosed herein, wherein said compositions comprise chitosan and betamethasone valerate, is shown below in Table 32 by considering various aspects of therapeutic cure of a compromised skin condition:

TABLE 32

| Therapeutic aspect | | Product of present invention |
|---|---|---|
| 1. | Diffusion study | The rate of release of Betamethasone Valerate |
| | | Cream of the invention was found to be 9.21 × |
| | | 10⁻⁵/hr when compared with reference product |
| | | shown 6.91 × 10⁻⁵/hr at the end of 9 hrs. |
| 2. | Skin Erythema | Betamethasone Valerate Cream of the invention |
| | study | (67.15%) had shown the highest percentage of |
| | | reduction in croton oil induced edema than refer- |
| | | ence product (66.79%). |
| 3. | Film forming | Yes (see FIG. 1) |
| | property | |
| 4. | Acute dermal | The study shows that the formulation of present |
| | irritation study | invention is non-irritant and dermal-friendly |
| | | when applied to the skin. |
| 5. | Clinical trial | Betamethasone Valerate Cream of the invention |
| | | had scored better than reference product based |
| | | on the various parameters such as VAS score, |
| | | GSI score, PGE score and Patient's compliance. |

121. It is evident from Table 32 summarizing the results of the study, that the film forming ability of the chitosan incorporated in the composition enables improved delivery of the API to infected area and results in better functioning, and importantly improved healing.

122. It is evident from the foregoing discussion that the present invention offers the following advantages and unique aspects over the currently available dermaceutical compositions for anti-inflammatory effect and pruritic manifestations of corticosteroid responsive dermatoses of the skin.
The compositions of the present invention include a skin-compatible biopolymer in the form of chitosan which enables enhanced therapeutic outcomes.
The compositions of the present invention uniquely incorporate a biopolymer without compromising the stability of the cream matrix and without adversely affecting the functioning of known active pharmaceutical ingredient. The resulting compositions unexpectedly achieve such results through a careful selection of functional excipients to bypass undesirable aspects of physio-chemical compatibility/stability and bio-release.
The compositions of the present invention provides an integrated unit-dose or a single-dose therapy hitherto unavailable in prescription dermaceutical formulations.
The novel compositions of the present invention are adequately stable/efficacious at ambient conditions and do not need special temperature control during transportation/storage.

123. The examples above are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### K. REFERENCES

1. Boucard N, Viton C, Agay D, Mari E, Roger T, Chancerelle Y, Domard A. The use of physical hydrogels of chitosan for skin regeneration following third-degree burns. Biomaterials. 2007; 28(24):3478-88.
2. Okamoto Y l, Shibazaki K, Minami S, Matsuhashi A, Tanioka S, Shigemasa Y. Evaluation of chitin and chitosan on open wound healing in dogs. J Vet Med Sci. 1995; 57(5):851-4.
3. The United States Pharmacopeia and National Formulary, USP 34-NF 29 Second Supplement Commentary. The United State Pharmacopeial Convention, Rockville. 2011.
4. Dai T, Tanaka M, Huang Y Y, Hamblin M R. Chitosan preparations for wounds and burns: antimicrobial and wound-healing effects. Expert Review of Anti-infective Therapy. 2011; 9(7):857-879.
5. The United States Pharmacopeia and National Formulary, USP 34-NF 29. Vol. 2. The United State Pharmacopeial Convention, Rockville. 2011.
6. PDR Staff. Physicians' Desk Reference, Medical Economics Company. 55^{th} Edition Medical Economics Data Production Company, 2000; p. A-350.
7. Lambers H, Piessens S, Bloem A, Pronk H, Finkel P. Natural skin surface pH is on average below 5, which is beneficial for its resident flora. International Journal of Cosmetic Science. 2006; 28(5):359-370.
8. Nguyen N, Hasan S, Caufield L, Ling F S, Narins C R. Randomized controlled trial of topical hemostasis pad use for achieving vascular hemostasis following percutaneous coronary intervention. Catheter. Cardiovasc. Interv. 2007, 69, 801-807.
9. Biagini B, Muzzarelli R A A, Giardino R, Castaldini C. Biological materials for wound healing. In: Brine C J, Sandford P A, Zikakis J P. Advances in chitin and chitosan, Vol. 1. 1992; p. 6-24.
10. Remington: The Science and Practice of Pharmacy. 19^{th} ed. A. R. Gennaro, Mack Publishing Company, Easton, 1995.

What is claimed is:

1. A composition comprising a topical corticosteroid and a biopolymer in a cream base, wherein the cream base comprises a primary and a secondary emulsifier, a waxy material, a co-solvent, a preservative, an acid, and water;
wherein the topical corticosteroid comprises betamethasone dipropionate or betamethasone valerate.

2. The composition of claim 1, further comprising a chelating agent, a buffering agent, an anti-oxidant, or a humectant.

3. The composition of claim 1, wherein betamethasone dipropionate is added in an amount between 0.001% (w/w) and 5% (w/w), between about 0.01% (w/w) and 2% (w/w), 0.12% (w/w) or at 0.064% (w/w).

4. The composition of claim 3, wherein the biopolymer comprises chitosan.

5. The composition of claim 4, wherein the chitosan is added in an amount between 0.01% (w/w) and 2% (w/w) by weight, in an amount from 0.01% (w/w) to 1.5% (w/w), or 0.5% (w/w).

6. The compositions of claim 5, wherein the chitosan has a molecular weight in the range of 50 kDa to 5000 kDa.

7. The composition of claim 6, wherein the primary and secondary emulsifiers are selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

8. The composition of claim 6, wherein the waxy material is selected from a group comprising white soft paraffin, liquid paraffin, and hard paraffin; and wherein the waxy material is added in an amount from 5% (w/w) to 30% (w/w).

9. The composition of claim 6, wherein the co-solvent is selected from a group comprising propylene glycol, hexylene glycol, polyethylene glycol-400; and wherein the co-solvent is added in an amount from about 5% (w/w) to 50% (w/w).

10. The composition of claim 6, wherein the acid is selected from a group comprising HCl, H₂SO₄, HNO₃, and lactic acid; and wherein the acid is added in an amount from about 0.005% (w/w) to 1% (w/w).

11. The composition of claim 6, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

12. The composition of claims 6, wherein the buffering agent is selected from the group comprising disodium hydrogen ortho phosphate, sodium hydrogen ortho phosphate; wherein the buffering agent is added in an amount of 0.05% (w/w) to 1% (w/w).

13. The composition of claim 6, wherein the water is purified water, and wherein the water is added in the range of 20% (w/w) to 75% (w/w), 35% (w/w) to 60% (w/w) or 40% (w/w) to 49% (w/w).

14. The composition of claim 6, further comprising anti-oxidants, wherein the anti-oxidant is selected from the group comprising butylated hydroxy anisole, or butylated hydroxy toluene; wherein the anti-oxidant is added in an amount of 0.001% (w/w) to 5% (w/w).

15. The composition of claim 6, further comprising a chelating agent, wherein the chelating agent is selected from the group comprising disodium EDTA; and wherein the chelating agent is added in an amount 0.05% (w/w) to 1% (w/w).

16. The composition of claim 5, further comprising a humectant, wherein the humectant is selected from a group comprising glycerin, propylene glycol, sorbitol; and wherein the humectant is added in an amount of 5% (w/w) to 20% (w/w).

17. A method for making a composition comprising the mixing of betamethasone dipropionate and a biopolymer in a cream base, wherein the cream base comprises a primary and a secondary emulsifier, a waxy material, a co-solvent, a preservative, an acid, and water.

18. The method of claim 17, further comprising an a chelating agent, a buffering agent, an anti-oxidant, or a humectant.

19. The method of claim 17, wherein betamethasone dipropionate is added in an amount between 0.001% (w/w) and 5% (w/w), between about 0.01% (w/w) and 2% (w/w), 0.12% (w/w) or at 0.064% (w/w).

20. The method of claim 17, wherein the biopolymer comprises chitosan.

21. The method of claim 20, wherein the chitosan is added in an amount between 0.01% (w/w) and 2% (w/w) by weight, in an amount from 0.01% (w/w) to 1.5% (w/w), or 0.5% (w/w).

22. The method of claim 21, wherein the chitosan has a molecular weight in the range of 50 kDa to 5000 kDa.

23. The method of claim 22, wherein the primary and secondary emulsifiers are selected from a group comprising cetostearyl alcohol, cetomacrogol-1000, cetyl alcohol, stearyl alcohol, isopropyl myristate, polysorbate-80, Span-80; and wherein the primary and secondary emulsifiers are present in the amount of 1% (w/w) to 25% (w/w).

24. The method of claim 22, wherein the waxy material is selected from a group comprising white soft paraffin, liquid paraffin, and hard paraffin; and wherein the waxy material is added in an amount from 5% (w/w) to 30% (w/w).

25. The method of claim 22, wherein the co-solvent is selected from a group comprising propylene glycol, hexylene glycol, polyethylene glycol-400; and wherein the co-solvent is added in an amount from about 5% (w/w) to 50% (w/w).

26. The method of claim 22, wherein the acid is selected from a group comprising HCl, H₂SO₄, HNO₃, and lactic acid; and wherein the acid is added in an amount from about 0.005% (w/w) to 1% (w/w).

27. The method of claim 22, wherein the preservative is selected from a group comprising methylparaben, propylparaben, chlorocresol, potassium sorbate, benzoic acid, phenoxyethanol, and benzyl alcohol; and wherein the preservative is added in an amount from 0.02% (w/w) to 0.5% (w/w).

28. The methods of claim 22, wherein the buffering agent is selected from the group comprising disodium hydrogen ortho phosphate, sodium hydrogen ortho phosphate; wherein the buffering agent is added in an amount of 0.05% (w/w) to 1% (w/w).

29. The method of claim 22, wherein the water is purified water, and wherein the water is added in the range of 20% (w/w) to 75% (w/w), 35% (w/w) to 50% (w/w) or 40% (w/w) to 49% (w/w).

30. The method of claim 20, further comprising anti-oxidants, wherein the anti-oxidant is selected from the group comprising butylated hydroxy anisole, butylated hydroxy toluene; wherein the anti-oxidant is added in an amount of 0.001% (w/w) to 5% (w/w).

31. The method of claim 20, further comprising a chelating agent, wherein the chelating agent is selected from the group comprising disodium EDTA; and wherein the chelating agent is added in an amount 0.05% (w/w) to 1% (w/w).

32. The method of claim 20, further comprising a humectant, wherein the humectant is selected from a group comprising glycerin, propylene glycol, sorbitol; and wherein the humectant is added in an amount of 5% (w/w) to 20% (w/w).

33. A method of treating skin problems comprising administering a composition wherein the composition comprises a topical corticosteroid and a biopolymer in a cream base, wherein the cream base comprises a primary and a secondary emulsifier, a waxy material, a co-solvent, a preservative, an acid, and water;
wherein the topical corticosteroid comprises betamethasone dipropionate or betamethasone valerate.

34. The method of claim 33, wherein the skin problem comprises a wound.

35. The method of claim 33, wherein the skin problem comprises acne, acne-related disorders, bacterial skin infections, skin tumors, bullous diseases, cancers of the skin, cornification disorders, fungal skin infections, hypersensitivity and inflammation, parasitic skin infections, pigmentation disorders, psoriasis, atopic dermatitis, eczema, contact dermatitis, dermatitis herpetiformis, generalized exfoliative dermatitis, seborrheic dermatitis, rosacea, shingles, sweating disorders, vitiligo and viral skin disease.
